(19) United States
(12) Patent Application Publication
(10) Pub. No.: US 2010/0055083 A1
(43) Pub. Date: Mar. 4, 2010

(54) ENCAPSULATION OF HEAT AND MOISTURE SENSITIVE SUBSTANCES

(76) Inventors: Ray Edward Kowalski, Morris plains, NJ (US); Shan-Shan Sheu, Randolph, NJ (US); Abdul Rashid, Livingston, NJ (US); Caroline Brons, Kinnelon, NJ (US); Elizabeth Nascimento, Warren, NJ (US)

Correspondence Address: NIXON & VANDERHYE, PC, 901 NORTH GLEBE ROAD, 11TH FLOOR, ARLINGTON, VA, 22203, US

(21) Appl. No.: 12/444,314

(22) PCT Filed: Oct. 18, 2007
(86) PCT No.: PCT/EP2007/009050

§ 371 Date: Nov. 5, 2009

Related U.S. Application Data
(60) Provisional application No. 60/852,429, filed on Oct. 18, 2006.

(51) Int. Cl. A61K 35/74; A61J 3/07

(52) U.S. Cl. 424/93.45; 424/93.4; 264/4

(57) ABSTRACT

The present invention relates to a method for preparing a suspension for the encapsulation of heat and moisture sensitive substances in capsules, sachets, droplets and food compositions. It also relates to methods for encapsulating, to the encapsulated products and to methods for storing the encapsulated products. The invention is extremely suitable for the encapsulation of microbial cultures. Cultures encapsulated in capsules prepared according to the method of the invention, will stay stable for a long time. At 25 degrees C. they show a stability reduction of less than 1 log per month.

### FIELD OF THE INVENTION

The present invention is in the field of encapsulated substances. In particular, it relates to heat and moisture sensitive substances for encapsulation. It also relates to encapsulated products and methods for encapsulation.

### BACKGROUND OF THE INVENTION

There are two types of capsules for encapsulation: soft shell and hard shell capsules. Soft shell capsules used to be for liquids and hard shell for solids, although recently a capsule has been developed for liquid storage in hard shell capsules and soft shell capsules now contain suspensions and solids.

Soft shell capsules are a very popular delivery form for dietary supplements worldwide, due to their ease of swallowing, appearance and preference, but they are not suitable for heat and moisture sensitive substances due to the way they are produced. Moisture and heat involved in their production may impair the stability of such substances. For example, hygroscopic substances are not very stable in soft shell capsules, because of the high water content.

Therefore, there is a need for more versatile preparations of heat and moisture sensitive substances which allow them to be encapsulated in soft shell capsules and at the same time does not significantly impair their stability.

### SUMMARY OF THE INVENTION

In a first aspect, the present invention relates to a suspension for encapsulation comprising:
(i) a heat and moisture sensitive substance for encapsulation, and
(ii) a non-aqueous filler in which the heat and moisture sensitive substance is suspended.

In another aspect, the invention relates to a capsule, sachet, droplet or food composition comprising a suspension according to the invention. In yet another aspect, the invention relates to the use of the non-aqueous filler or carrier material in a method for preparing a suspension for encapsulation of a heat and moisture sensitive substance. In yet another aspect, the present invention relates to a container comprising a soft gel capsule according to the invention. In yet another aspect, the present invention relates to a method for producing a soft gel capsule containing a heat and moisture sensitive substance.

Accordingly, the invention also provides:
a method for preparing a suspension for encapsulation of a heat and moisture sensitive substance which method comprises mixing a non-aqueous filler as defined herein with a heat and moisture sensitive substance;
a capsule, sachet, droplet or food composition comprising a suspension of the invention;
a capsule consisting of a shell which surrounds a matrix, wherein the matrix comprises a suspension of the invention, wherein the suspension is a microbial liquid suspension, and which is characterised in that it has less than about 1 log reduction of CFU per month at 25 degrees C.
a container comprising a capsule of the invention, wherein the head space of the container has a relative humidity which is less than or equal to about 30%.
a method for producing a capsule, which method comprises:
mixing a heat and moisture sensitive substance with a non-aqueous filler to obtain a suspension;
encapsulating the suspension in a capsule or shell;
drying the resulting capsules and storing them in a container with an internal relative humidity of less than 30%;
a method for producing a soft gel capsule:
mixing a heat and moisture sensitive substance with a non-aqueous filler to obtain a suspension;
encapsulating the suspension in a soft gel capsule or soft shell;
drying the soft gel capsules and storing them in a container with an internal relative humidity of less than 30%;
a method for stably storing a capsule, for example a capsule of the invention (such as a softgel capsule of the invention), with less than about 1 to about 2 log reduction per month at about 25 degrees C., wherein the method comprises: storing the capsules in a container with an internal relative humidity of about 30% or less; and
the use of a suspension, a capsule, sachet, droplet, food composition or a container of the invention in the food, feed, cosmetic or nutritional supplement industry.

### DETAILED DESCRIPTION OF THE INVENTION

In a first aspect, the present invention relates to a suspension for encapsulation comprising:
(ii) a heat and moisture sensitive substance for encapsulation, and
(iii) a non-aqueous filler in which the heat and moisture sensitive substance is suspended.

The heat and moisture sensitive substances may be in any suitable form, be it solid or liquid form, be it a gel, powder or a granulate.

In the context of the present invention, the terms “suspension” and “mixture” are used interchangeably. They both refer to the situation that two or more substances are mixed. It includes a fine dispersed mixture of a solid in a liquid. It also includes bacteria in a liquid. The suspension or mixture may be homogeneous or heterogeneous.

The term “encapsulation” refers to the technology of packaging a substance within another material. In the context of the present invention, the material which has been entrapped is referred to as “matrix” or “core”. In this context, the matrix or core comprises the heat and moisture sensitive substance suspended in a non-aqueous filler or carrier material. The surrounding material is referred to as “shell”.

In the present context, the phrase “heat and moisture sensitive substances” refers to substances which are likely to be partly or completely damaged and will not work or function properly or to their fullest extent after exposure to elevated temperatures and/or moisture. Examples of heat and moisture sensitive substances which may suitably be used in the suspensions according to the invention include hygroscopic substances, hydrophilic substances, such as calcium chloride, magnesium chloride, potassium iodide; microorganisms, such as yeast and bacteria, probiotics, starter cultures; essential nutrients, such as vitamins; pharmaceuticals, neutraceuticals, dietary supplements, such as prebiotics, such as inuline, fructo-oligosaccharides.

If the heat and moisture sensitive substance is a microbial culture, it is preferably a dried microbial culture. The culture may be dried by any method known in the art, such as vacuum drying, freeze-drying, spray-drying, fluidized-bed drying or desiccant absorption or any combination thereof.

In one embodiment of the invention, the heat and moisture sensitive substance is a probiotic or a mixture of probiotics. In the context of the present invention, a probiotic is defined as a live microbial strain, which beneficially affects the human host cell by improving its microbial balance. Preferred probiotics are isolated strains of Bifidobacterium, Lactobacillus, Propionibacterium, Enterococcus, and mixtures of two or more thereof. More preferred probiotic species are Bifidobacterium infantis, Bifidobacterium longum, Bifidobacterium animalis, Bifidobacterium lactis, Lactobacillus acidophilus, Lactobacillus rhamnosus, Lactobacillus casei, Lactobacillus paracasei, Lactobacillus helveticus, and mixtures thereof. According to a preferred embodiment, the heat and moisture sensitive substance is one of the following probiotics: Lactobacillus acidophilus strain LAFTI® L10 deposited at the Centraalbureau voor Schimmelcultures (CBS) under accession number CBS 116411, Lactobacillus casei strain LAFTI® L26 deposited at the CBS under accession number CBS 116412 and LAFTI® B94, which is a Bifidobacterium animalis deposited at the CBS under the accession number CBS 118529. According to a preferred embodiment, the probiotic consists of a biologically pure culture or substantially biologically pure culture of at least said deposited strain(s). According to a more preferred embodiment, the probiotic consists of a biologically pure culture or substantially biologically pure culture of at least said deposited strain(s) in combination with any other valuable probiotic. According to an even more preferred embodiment, the probiotic is selected from the group consisting of Bifidobacterium infantis, Bifidobacterium longum, Bifidobacterium lactis, Bifidobacterium animalis, Lactobacillus acidophilus, preferably Lactobacillus acidophilus LAFTI® L10 CBS 116411, Lactobacillus rhamnosus, Lactobacillus casei, preferably Lactobacillus casei LAFTI® L26 CBS 116412, Lactobacillus paracasei and Lactobacillus helveticus. Even more preferably, the probiotic is Lactobacillus acidophilus CBS 116411 or Lactobacillus casei CBS 116412 or a Bifidobacterium animalis CBS 118529, or mixtures thereof. Accordingly, the heat and moisture sensitive substance may comprise a mixture or two or more of the deposited strains mentioned above.

Non-aqueous filler or carrier material which may suitably be used in the suspension according to the invention includes:

waxes, such as beeswax, paraffin wax, animal stearates, solid glycol esters, mono and diglycerides and wax mixtures;

oils, such as olive oil, sunflower oil, canola oil, coconut oil, corn oil, palm oil, sesame oil, peanut oil, along with medium chain triglycerides;

an emulsifier, for example a phospholipid such as lecithin;

a glyceride such as a mono- or di-glyceride or a combination thereof, for example a stearate, for example glycerol monostearate, such as an animal or vegetable stearate, and/or a palmitate such as glycerolmonopalmitate.

a dispersant or a surfactant such as polyethylene glycols.

They may be used as such, but also combinations of different non-aqueous filler materials may be used.

One of more, for example two, three, four, five or all, of an excipient, a desiccant, a nutrient, a prebiotic, an enzyme and a vitamin may also be added to the matrix.

A suspension of the invention will typically have a viscosity sufficient to prevent segregation during processing and/or which also provides adequate protection from air/moisture entrapment.

In one embodiment of the invention, a suspension according to the invention may comprise from about 5 to about 80 w/w % of the heat and moisture sensitive substance, from about 10 to about 50 w/w % beeswax, from about 5 to about 60 w/w % sunflower oil and from about 0.1 to about 5 w/w % lecithin. In a preferred embodiment, a suspension is used which comprises from about 5 to about 40 w/w % of the heat and moisture sensitive substance, from about 20 to about 45 w/w % beeswax, from about 20 to about 45 w/w % sunflower oil and from about 0.1 to about 3 w/w % lecithin.

In another preferred embodiment of the invention, a suspension according to the invention comprises a heat and moisture sensitive substance and a non-aqueous filler which comprises one or more selected from the group which consists of a wax, an oil, an emulsifier and a glyceride.

More specifically, the non-aqueous filler may comprise one or more selected from the group which consists of beeswax, sunflower oil, lecithin, a monoglyceride, a diglyceride and a mixture of a mono- and di-glyceride.

The suspension according to the invention may comprise:

from about 0.5 to about 80 w/w %, such as of the heat and moisture sensitive substance, such as from about 10 to about 50 w/w %, for example from about 25 to about 40 w/w %

from about 0.1 to about 20 w/w % of a wax, such as from about 1 to about 10 w/w %, for example from about 1 to about 5 w/w %;

from about 5 to about 90 w/w % of an oil, such as from about 20 to about 70 w/w %, for example from about 40 to about 60 w/w %;

from about 1 to about 30 w/w % mono- and/or di-glycerides, such as from about 5 to about 20 w/w %, for example from about 5 to about 15 w/w %; and

from about 0.1 to about 10 w/w % of an emulsifier, such as from about 0.5 to about 5 w/w %, for example about 1 to about 5 w/w %.

Accordingly, the suspension according to the invention may comprise from about 0.5 to about 80 w/w % of the heat and moisture sensitive substance (eg, from about 10 to about 50 w/w %, such as from about 25 to about 40 w/w %), from about 0.1 to about 20 w/w % beeswax (eg. from about 1 to about 10 w/w %, such as from about 1 to about 5 w/w %), from about 5 to about 90 w/w % sunflower oil (eg. from about 20 to about 70 w/w %, such as from about 40 to about 60 w/w %), from about 1 to about 30 w/w % mono- and/or di-glycerides, in particular monoglycerides (eg. from about 5 to about 20 w/w %, such as from about 5 to about 15 w/w %) and from about 0.1 to about 10 w/w % lecithin (eg. from about 0.5 to about 5 w/w %, such as from about 1 to about 5 w/w %).

The suspension according to the invention may comprise any combination of the amounts given above.

In yet another aspect, the invention relates to the use of the non-aqueous filler or carrier material in a method for preparing a suspension for encapsulation of a heat and moisture sensitive substance. This method comprises mixing a non-aqueous filler material as described above with a heat and moisture sensitive substance. In a preferred embodiment, the heat and moisture sensitive substance is a microbial culture, more preferably a probiotic culture or a starter culture.

In another aspect, the invention relates to a capsule, sachet, droplet or food composition comprising a suspension according to the invention. In a preferred embodiment, the invention relates to a food composition with high amounts of oil, such as for example margarine, spread, butter, salad oil, chocolate or couvertures.

In one embodiment, the present invention relates to a capsule consisting of a shell which surrounds a matrix and wherein the matrix comprises a suspension according to the invention.

The shell of the capsule may be a soft shell or a hard shell made from material known in the art and includes film formers such as soft shell film formers based on gelatine or copolymers with other biopolymers, such as pectin and cellulose. It includes film formers such as hard shell film formers based on gelatine, hydroxypropyl methyl cellulose (HPMC), carrageenan, rice protein, tapioca starch and commonly used film formers. Typically, capsule shells contain additional ingredients such as plasticizers, such as for example glycerine and sorbitol, water, preservatives, colourants, opacifiers, which are all known ingredients to the person skilled in the art (see also Stanley, J. P., “Part Two. Soft Gelatin Capsules,” in The Theory and Practice of Industrial Pharmacy, Lachman, L., et al., eds., Lea & Febiger, Philadelphia, Pa., pp. 398-412 (1986).

In one embodiment of the invention, the capsule according to the invention may contain a liquid suspension according to the invention. Suspensions which are wholly or at least partially liquid at or above room temperature and become solid at room temperature or lower temperatures are also encompassed by the present invention.

In another embodiment of the invention, the capsule according to the invention is a hard shell capsule. The hard shell capsule according to the invention may comprise a suspension according to the invention in any suitable form.

The heat and moisture sensitive substances may also be packaged in soft shell capsules according to the invention. Normally, a soft shell capsule would create an environment with a water activity (a_{w}) which is too high for these kinds of substances. Therefore, in another embodiment of the invention, the capsule according to the invention is a soft shell capsule. The soft shell capsule according to the invention may encapsulate a suspension according to the invention in any suitable form.

In yet another embodiment of the invention, a capsule, such as a soft shell capsule, has been dried to a water activity of less than 0.3. In a preferred embodiment of the invention, the capsule, such as a soft shell capsule, has been dried to a water activity of less than about 0.28, about 0.26, about 0.24, about 0.22 or about 0.20. More preferably, it is dried to a water activity of less than about 0.18, about 0.16, about 0.14, about 0.12 or about 0.10. Most preferably, the soft shell capsules are dried to a water activity of less than about 0.09, about 0.08, about 0.07, about 0.06, about 0.05 or about 0.04. In a preferred embodiment, the soft shell has been dried to a water activity of about 0.25 or less or about 0.23 or less. The soft shell capsules according to the invention are firm, but not brittle or fragile. This has several advantages, for example during handling, packaging or transportation.

In a preferred embodiment of the invention, the soft shell capsules are soft shell capsules based on gelatin.

In another embodiment of the invention, the capsule contains a matrix with a microbial culture in the suspension and is characterised in that is has less than 1 log reduction in colony forming units (CFU) per month at 25 degrees C. Methods for determining CFU are well known to those skilled in the art. In the context of this invention, CFU is typically determined by use of the DSM LAFTI L10 enumeration method which is available on request and which is set out as follows:

1. Determine how many dilutions are required based on the estimated viability of the original sample. Typically, there should be a range of 30 to 300 colonies on the final culture plate. Usually this amount of colonies is found in the 10⁻⁷, 10⁻⁸, 10⁻⁹ or 10⁻¹⁰ dilution.

2. Preparation of dilution fluids:

Dissolve the following chemicals in 1 liter of sterile demineralised water:

0.5 grams of L-Cysteine (0.05%)

4.5 grams of KH₂PO₄ (0.45%)

6.0 grams of Na₂HPO₄ (0.6%)

Adjust the pH of the solution to 7.4 using a 4M NaOH solution.

A part of the solution prepared above is adjusted to pH 9, using 4M NaOH. This solution is used for the first dissolution step of a dissolution series.

3. Preparation of dilution series:

Fill one tube of the dilution series with 9.0+/−0.2 ml of solution fluid at pH 9. Fill all other tubes with 9.0+/−0.2 ml of solution at pH 7.4.

Close all 9.0+/−0.2 ml tubes to prevent evaporation of moisture and exclude aerial infection using a cap or a marble.

Dose 1.00+/−0.2 ml gram of microbial culture in the dilution fluid at pH 9. Vortex and shake the tube until a visually homogenous suspension is obtained.

Pipette 1 ml of this suspension into a tube with dissolution fluid at pH 7.4. Vortex until the suspension is homogenous. (1-3 seconds).

Repeat pipetting 1 ml of this suspension into a tube with dissolution fluid at pH 7.4 and vortexing until the desired dilution is reached, usually 10⁻⁷, 10⁻⁸, 10⁻⁹ and 10⁻¹⁰.

4. Incubating:

Pipette 1.0 ml of the desired dilution in a culture plate (petridish). Pour 15-20 ml of sterile MRS agar at a temperature of approximately 50 degrees Celsius into the petridish. First close and then swirl the petridish gently in order to spread the material throughout the agar.

Allow the agar to solidify, and incubate under anaerobic conditions at 37 degrees Celsius for three days.

Count the number of formed colonies on the culture plate, and calculate the number of CFU/g using the chosen dilution.

Recommended MRS-agar is commercially available via Becton, Dickinson and company: Difco™ Lactobacilli MRS Agar, Productmunmber: 288210.

A 1 log reduction in CFU is for instance a reduction from 1×10⁹ to 1×10⁸ (from 1E+9 to 10E+8) or from 1×10⁵ to 1×10⁴ (from 1E+5 to 1E+4). Preferably, an average of about 0.5 log reduction in CFU or less per month at 25 degrees C. is achieved. This is preferably observed in the first few months after production. Most preferably in the first 3 months after production of the capsules. Even greater stability is possible by storage at lower temperatures such as refrigerated conditions, such as from about 2 to about 10° C.

In yet another aspect, the present invention relates to a container comprising a capsule, for example a soft gel capsule, according to the invention, wherein the head space of the container has a relative humidity which is about 30% or less. It may take days or weeks to reach a relative humidity of 30%. The time required to reach a steady state of 30% will depend several factors, such as on the composition of the capsule, the type of desiccant used, the size of the bottle, the number of capsules in the bottle and the size of the head space. In a preferred embodiment of the invention, a relative humidity of less than 30% is reached in two days, most preferably by using dry air.

Such relative humidity may be reached by any method known to the skilled in the art for reducing the relative humidity, such as by forced drying and absorption drying and includes the use of desiccants. Suitable examples are known in the art, see for example Handbook of Pharmaceutical Excipients, 3^{rd} ed. by Arthur H. Kibbe 2000 APhA (American Pharmaceutical Association) and PhP (Pharmaceutical Press), and include calcium sulphate, sodium sulphate, silicon dioxide.

In yet another aspect, the present invention relates to a method for producing a capsule, such as a soft gel capsule, which encapsulates a heat and moisture sensitive substance comprising:

mixing a heat and moisture sensitive substance with a non-aqueous filler, such as a filler as described above to obtain a suspension, for example a suspension according to the invention, for encapsulation;

encapsulating the suspension in a capsule, such as a soft gel or hard shell capsule;

drying the soft gel capsules to a water activity of the shell of less than 0.3, and

storing the dried capsules in an environment with an internal relative humidity of less than 30%.

The storage conditions may include storage at room temperature, such as at a temperature of from about 20 to about 27° C. and also includes refrigerated conditions, such as from about 2 to about 10° C.

The mixing and/or encapsulation may take place in a low humidity environment.

In the context of the present invention, the phrase “low humidity environment” refers to an environment with a relative humidity of less than 50%. Preferably, it refers to an environment with a relative humidity of less than 30%. More preferably, the relative humidity is in the range of 10-30%, more preferably in the range of 10-15%. Low humidity may be reached by any method known to the skilled in the art for reducing the relative humidity, and includes the use of desiccants, dry compressed air, cooling, air conditioning, LiCl and nitrogen purging.

Any sample for encapsulation, for example soft gel encapsulation, first has to conform to the standard particle size spec requirement (100% through 80 mesh) requested by most soft shell gelatin capsule manufacturers for products intended to be encapsulated. This requirement is necessary to prevent problems (improper gelatin seal or leaks) during capsule filling. Therefore the mixing step may be preceded by a size reduction step which should also be carried out in a low humidity environment. For size reduction, a method which does not damage the heat and moisture sensitive substance should be used, for example sieving or oscillating. It is important that the particles are reduced to such an extent that they will pass through an injection needle, which is the usual method for filling soft shell capsules. In a preferred embodiment of the invention at least 95% of the particles of the size-reduced heat and moisture sensitive substance fall through 80 mesh.

The heat and moisture sensitive substance according to the invention may be mixed with any non-aqueous filler, such as waxes, such as beeswax, paraffin wax, animal stearates, solid glycol esters, mono and diglycerides and wax mixtures; oils, such as olive oil, sunflower oil, canola oil, coconut oil, corn oil, palm oil, sesame oil, peanut oil, along with medium chain triglycerides; lecithin; polyethylene glycols, solid glycol esters, animal stearates. They may be used as such, but also combinations of different non-aqueous filler materials may be used. Preferably, the non-aqueous filler comprises beeswax, sunflower oil and lecithin and, preferably, mono- and/or di-glycerides (in particular, monoglycerides). The non-aqueous filler may be as described above. Excipients, desiccants, nutrients, prebiotics and vitamins may also be added to the heat and moisture sensitive substance containing matrix.

Any drying method known in the state of the art may be used for drying the capsules which contain the heat and moisture sensitive substance. In a preferred embodiment of the invention, the capsules are dried by tumble drying at for example about 25 degrees C. for from about 10 to about 40 minutes, followed by tray drying at room temperature, for example about 25° C., or lower for at least about 1 or 2 to about 14 days. The relative humidity may be about 50% or less.

An alternate and/or additional approach to remove water would be to use a different soft gel gelatine capsule formulation with initially lower water prior to any additional drying.

The capsules, in particular soft shell capsules, may be further dried by using a desiccant, by vacuum tray drying, fluidised bed drying, infrared or by dehydrated air.

The dried capsules are stored in an environment with a relative humidity about 30% or less to prevent an increase of the water activity of the carefully prepared capsules. Preferably, the capsules are stored in an environment with a relative humidity of less than about 28% or less, about 26% or less, about 24% or less, about 22% or less or about 20% or less. More preferably, they are stored in an environment with a relative humidity of about 18% or less, about 16% or less, about 14% or less, about 12% or less or about 10% or less. Most preferably, they are stored in an environment with a relative humidity of about 9% or less, about 8% or less, about 7% or less, about 6% or less, about 5% or less or about 4% or less.

In one preferred embodiment of the invention, the dried capsules are stored in a container of which the headspace has an internal relative humidity of 30% or less. This relative humidity may have been reached rapidly after filling the container with capsules, for example by the addition of desiccants in the container. The same container may conveniently be used for handling, packaging and transportation to end users. Containers may also be stored at refrigerated temperatures, such as at about 2 to 10° C., in order to maintain the desired stability.

Desiccants suitable for use in the invention include any suitable desiccant such as silica, silica gel or zeolites.

This process of the invention yields soft shell capsules which are very stable. In a preferred embodiment, soft shell capsules encapsulating live microorganisms are produced this way. The capsules show less than 1 log reduction of colony forming units per month at 25 degrees C. Therefore, the present invention also encompasses a method for the stable storage of a soft gel capsule containing live microorganisms with less than about 1 log reduction per month at 25 degrees C., or even better stability at lower temperatures such as refrigerated conditions (for example from about 2 to about 10° C.), wherein the method comprises:
optionally subjecting a heat and moisture sensitive substance to particle size reduction;
mixing the size-reduced substance with a non-aqueous filler to obtain a suspension for encapsulation;
encapsulating the suspension in a soft gel capsule;

wherein, optionally, one or more of particle size reduction (if carried out), mixing and encapsulation take place in a low humidity environment;
drying the soft gel capsules to a water activity of the shell of about 0.3 or less, and
storing the dried capsules in an environment with an internal relative humidity of about 30% or less.

Greater stability may be obtained by storage at a lower temperature, such as refrigerated condition, for example from about 2 to about 10° C.

For example, stability of about 0.5 log reduction in CFU or less per month, such as about 0.3 log reduction in CFU or less per month or about 0.1 log reduction in CFU or less per month may be achieved using the methods described herein. In particular when a lower temperature, such as from about 2 to about 10° C. is used. At about 4° C., about 1 log reduction in CFU or less per 6 months may be achieved.

The internal relative humidity of about 30% or less is preferably achieved by storing the capsules in the presence of desiccants, such as one or more of those described above, immediately after production of the capsules.

In another aspect, the invention relates to a capsule, sachet, droplet or food composition comprising a suspension according to the invention may find use as a nutraceutical, i.e. a pharmaceutical product that has nutritional value or a food that has its nutritional value enhanced by a pharmaceutical (for example the microorganism) or as a nutritional ingredient, or as a health ingredient, or as a supplement to food or beverage or as a stand-alone medicament.

The following Examples illustrate the invention:

### EXAMPLES

### Example 1

### Preparation of a Probiotic Sample in a Suspension According to the Invention

A probiotic bacteria sample of coated granules of freeze dried Lactobacillus acidophilus (Lafti L10, DSM, Delft, the Netherlands) was subjected to particle size reduction by passing the probiotic sample through a lab scale Oscillator equipped with a fine screen. The entire operation was enclosed in a plastic bag purged with compressed air to achieve low relative humidity (10-15% RH) to prevent potency loss during processing. Several filler materials were prepared. In all cases the filler material comprised 5-60% sunflower oil, 10-50% beeswax and 0.1-5% lecithin. This was mixed with bacteria to obtain an end concentration of 5-80% of bacteria. If necessary, a gentle mixing step was applied prior to encapsulation to keep the mixture of filler material and bacteria evenly suspended to ensure good Lafti L10 content uniformity in the capsules. This could also be solved by increasing the viscosity. In that case, the suspension showed less tendency to segregate and was more uniformly distributed, resulting in a better processability. Suspensions were left at room temperature for 3 months.

A representative result is shown in Table 1 and it is clear that the Lafti L10 is stable in the suspension medium according to the invention. The CFU recovery stays literally unchanged. This shows that carriers as defined in the invention can be used to prepare a stable dispersion of a microbial suspension.

TABLE 1

| Stability of a suspension according to the | |
|---|---|
| invention (storage at room temperature) | CFU (colony forming units) |
| Raw material | 1.80E+10 |
| Suspension | 1.41E+10 |
| 0 months | 1.41E+10 |
| 1 month | 1.68E+10 |
| 2 months | — |
| 3 months | 1.68E+10 |

### Example 2

### Preparation of a Probiotic Sample in a Suspension According to the Invention

A probiotic bacteria sample of coated granules of freeze dried Lactobacillus acidophilus (Lafti L10, DSM, Delft, the Netherlands) was subjected to particle size reduction by passing the probiotic sample through a lab scale Oscillator equipped with a 80 mesh screen. The entire operation was enclosed in a plastic bag purged with compressed air to achieve low relative humidity (10-15% RH) to prevent potency loss during processing.

Several filler materials were prepared. In all cases the filler material comprised 5-90 w/w % sunflower oil, 0.1-20 w/w % beeswax, 1-30 w/w % monoglycerides and 0.1-10 w/w % lecithin. This was mixed with bacteria to obtain an end concentration of 5-80% of bacteria. If necessary, a gentle mixing step was applied prior to encapsulation to keep the mixture of filler material and bacteria evenly suspended to ensure good Lafti L10 content uniformity in the capsules. This could also be solved by increasing the viscosity as in Example 4. In that case, the suspension showed less tendency to segregate and was more uniformly distributed, resulting in a better processability. Suspensions were left at room temperature for 3 months.

A representative result is shown in Table 2 and it is clear that the Lafti L10 is stable in the suspension medium according to the invention. The CFU recovery stays literally unchanged. This shows that carriers as defined in the invention can be used to prepare a stable dispersion of a microbial suspension.

Formulation 1

| | milligrams | % |
|---|---|---|
| Lafti L10 | 80.0 | 28.6 |
| Beeswax | 7.2 | 2.6 |
| Sunflower Oil | 156.0 | 55.7 |
| Monoglycerides | 30.0 | 10.7 |
| Lecithin | 6.8 | 2.4 |

TABLE 2
(Stability of suspension - formulation 1)

| Stability of a suspension according to the | |
|---|---|
| invention (storage at room temperature) | CFU (colony forming units) |
| Raw material | 1.80E+10 |
| Suspension | 1.41E+10 |
| 0 months | 1.41E+10 |
| 1 month | 1.68E+10 |
| 3 months | 1.68E+10 |
| 6 months | 1.71E+10 |

### Example 3

### Preparation of Capsules and their Stability

A suspension according to Example 1 was encapsulated in soft gelatin capsules at a relative humidity of up to 41.6% and a temperature of about 25 degrees C. Capsules were stored in HDPE plastic bottles and silicagel was added as a desiccant directly to the Lafti L10 capsules in the bottles. The bottles were stored at 25° C. A parallel experiment was run in which no desiccants were added to the bottle with capsules. Results for 3-month storage are given in Table 3. The results show that good results are obtained using the method and suspension of the invention, because the CFU reduction is less than 0.3 log per month.

TABLE 3

| Stability of an encapsulated | | |
|---|---|---|
| suspension according to the | CFU | CFU |
| invention (storage at room | When stored | When stored |
| temperature) | with desiccant | without desiccant |
| Raw material | 1.44E+10 | 1.44E+10 |
| Suspension | 1.40E+10 | 1.40E+10 |
| 0 months | 1.04E+10 | 1.04E+10 |
| 1 month | 7.89E+09 | 1.06E+09 |
| 2 months | 2.66E+09 | 7.84E+07 |
| 3 months | 2.74E+09 | 8.96E+06 |

### Example 4

### Preparation of Capsules and their Stability

A suspension according to Example 2 was encapsulated in soft gelatine capsules at a relative humidity of up to 42% and a temperature of about 25 degrees C. Capsules were stored in HDPE plastic bottles and silica gel was added as a desiccant directly to the Lafti L10 capsules in the bottles. The bottles were stored at 25 degree C. A parallel experiment was run in which no desiccants were added to the bottle with capsules. Results for 18-month storage are given in Table 4. The results show that good results are obtained using the method and suspension of the invention, because the CFU reduction is less than 0.3 log per month.

TABLE 4

| Stability of encapsulated | | |
|---|---|---|
| softgel capsules according | CFU | CFU |
| to the invention (storage | When stored | When stored |
| at room temperature) | with desiccant | without desiccant |
| Raw material | 1.44E+10 | 1.44E+10 |
| Suspension | 1.40E+10 | 1.40E+10 |
| 0 months | 1.04E+10 | 1.04E+10 |
| 1 month | 2.66E+09 | 1.06E+09 |
| 2 months | 2.74E+09 | 7.84E+07 |
| 3 months | 2.18E+09 | 8.96E+06 |
| 4 months | 1.96E+09 | |
| 6 months | 3.09E+07 | 3.30E+04 |
| | | (stopped at 6M) |
| 12 months | 1.10E+05 | *at 4° C. (4.00E+09) |
| 18 months | 9.5E+06 | *at 4° C. (4.00E+09) |

### Example 5

### Preparation of Capsules and their Stability

### Another Formulation was Tested at Various % of Each Ingredient

A formulation was prepared as set out in the table below and tested when stored with desiccant (MiniPax™ pouch from Multisorb Technologies, Inc. containing silicagel.) at room temperature (25° C.) as compared with 4° C.

Formulation 2

| | milligrams | % |
|---|---|---|
| Lafti L10 | 100.0 | 35.7 |
| Beeswax | 8.4 | 3.0 |
| Sunflower Oil | 129.5 | 46.2 |
| Monoglycerides | 35.0 | 12.5 |
| Lecithin | 7.1 | 2.6 |

The results are set out in Table 5. A beneficial effect may be seen for lowered temperature.

TABLE 5

| Stability of encapsulated | CFU | CFU |
|---|---|---|
| softgel capsules according | When stored | When stored |
| to the invention (storage | with desiccant | with silicagel |
| at room temperature) | at RT (25° C.) | desiccant at 4° C. |
| Raw material | 2.00E+10 | 2.00E+10 |
| Suspension | 1.82E+10 | 1.82E+10 |
| 0 months | 8.40E+09 | 8.4E+09 |
| 1 month | 3.10E+09 | |
| 2 months | 4.17E+09 | |
| 3 months | 1.74E+09 | |
| 4 months | 6.20E+08 | |
| 6 months | 8.96E+07 | 5.88E+09 |
| 12 months | 5.70E+08 | 3.60E+09 |

1. A suspension for encapsulation comprising
(i) a heat and moisture sensitive substance for encapsulation, and
(ii) a non-aqueous filler in which the heat and moisture sensitive substance is suspended.

2. A suspension according to claim 1, wherein the non-aqueous filler comprises one or more selected from the group which consists of a wax, an oil and an emulsifier.

3. A suspension according to claim 1, wherein the non-aqueous filler comprises one or more selected from the group which consists of a wax, an oil, an emulsifier and a glyceride.

4. A suspension according to claim 1, wherein the non-aqueous filler comprises one or more selected from the group which consists of beeswax, sunflower oil and lecithin.

5. A suspension according to claim 1, wherein the non-aqueous filler comprises one or more selected from the group which consists of beeswax, sunflower oil, lecithin, a monoglyceride, a diglyceride and a mixture of a mono- and di-glyceride.

6. A suspension according to claim 1, which comprises from about 0.5 to about 80 w/w % of the heat and moisture sensitive substance, from about 0.1 to about 20 w/w % beeswax, from about 5 to about 90 w/w % sunflower oil, from about 1 to about 30 w/w % mono- and/or di-glycerides and from about 0.1 to about 10 w/w % lecithin.

7. A suspension according to claim 1, which comprises from about 5 to about 80 w/w % of the heat and moisture sensitive substance, from about 10 to about 50 w/w % beeswax, from about 5 to about 60 w/w % sunflower oil and from about 0.1 to about 5 w/w % lecithin.

8. A suspension according to claim 1, wherein the heat and moisture sensitive substance comprises a microbial culture.

9. A suspension according to claim 1, wherein the microbial culture is a starter culture or a culture of probiotic bacteria.

10. A suspension according to claim 1, wherein the probiotic bacteria belong to a genus selected from the group consisting of Lactobacillus and Bifidobacterium.

11. A method for preparing a suspension for encapsulation of a heat and moisture sensitive substance which method comprises mixing a non-aqueous filler as defined in claim 1 with a heat and moisture sensitive substance.

12. A capsule, sachet, droplet or food composition comprising a suspension according to claim 1.

13. A food composition according to claim 12 wherein the food composition is a high oil containing product.

14. A capsule consisting of a shell which surrounds a matrix, wherein the matrix comprises a suspension according to claim 1, wherein the suspension is a microbial liquid suspension, and which is characterised in that it has less than about 1 log reduction of CFU per month at 25 degrees C.

15. A capsule according to claim 14, wherein the suspension is a microbial liquid suspension, and which is characterised in that it has less than about 0.1 log reduction of CFU per month at from about 2 to about 10 degrees C.

16. A capsule according to claim 12, wherein the capsule is a hard shell capsule.

17. A capsule according to claim 12, wherein the capsule is a soft shell or soft gel capsule.

18. A capsule according to claim 12 wherein the capsule has been dried to a water activity of about 0.3 or less.

19. A container comprising a capsule according to, wherein the head space of the container has a relative humidity which is less than or equal to about 30%.

20. A container according to claim 19 which comprises a desiccant.

21. A method for producing a capsule, which method comprises:
mixing a heat and moisture sensitive substance with a non-aqueous filler to obtain a suspension;
encapsulating the suspension in a capsule or shell; —drying the resulting capsules and storing them in a container with an internal relative humidity of less than 30%.

22. A method for producing a soft gel capsule:
mixing a heat and moisture sensitive substance with a non-aqueous filler to obtain a suspension;
encapsulating the suspension in a soft gel capsule or soft shell;
drying the soft gel capsules and storing them in a container with an internal relative humidity of less than 30%.

23. A method according to claim 21, wherein the non-aqueous filler is a suspension for encapsulation comprising
(i) a heat and moisture sensitive substance for encapsulation, and
(ii) a non-aqueous filler in which the heat and moisture sensitive substance is suspended.

24. A method according to claim 1, wherein the method is for producing a soft gel capsule.

25. A method according to claim 21, wherein mixing and encapsulation take place in a low humidity environment.

26. A method according to claim 21, wherein the mixing step is preceded by a size reduction step.

27. A method according to claim 26, wherein at least 95% of the particles of the size-reduced heat and moisture sensitive substance falls through 80 mesh.

28. A method according to claim 21, wherein the capsules are dried to reach a water activity of about 0.3 or less.

29. A method according to claim 21, wherein the capsules are further dried by using a desiccant, by vacuum tray drying, fluidised bed drying, by tumble drying or by dehydrated air with or without a soft gel shell formula low water content formula selection.

30. A method for stably storing a capsule with less than about 1 to about 2 log reduction per month at about 25 degrees C., wherein the method comprises: storing the capsules in a container with an internal relative humidity of about 30% or less.

31. A method according to claim 30, wherein an internal relative humidity of less than 30% is achieved by storing the capsule in the presence of desiccants immediately after production of the capsules.

32. A method according to claim 30, wherein the capsule is stored in a container at a temperature of from about 2 to about 10° C.

33. A method according to claim 30, wherein the capsule is a capsule comprising a suspension.

34. A method according to claim 30, wherein the capsule is a soft gel capsule.

35. A method according to claim 34, wherein the capsule is a soft gel capsule.

36. The use of a suspension according to claim 1, a capsule, sachet, droplet or food composition in the food, feed, cosmetic or nutritional supplement industry.
